(19) United States
(12) Patent Application Publication
(10) Pub. No.: US 2014/0276793 A1
(43) Pub. Date: Sep. 18, 2014

(54) HANDPIECE FOR AN ABLATION DEVICE HAVING TOOL PIECE WITH MULTIPLE ORIENTATIONS AND METHOD FOR RECONFIGURING HANDPIECE

(71) Applicant: Eliot F. Bloom, Hopkinton, NH (US)

(72) Inventor: Eliot F. Bloom, Hopkinton, NH (US)

(73) Assignee: MEDTRONIC ADVANCED ENERGY LLC, Minneapolis, MN (US)

(21) Appl. No.: 13/795,956

(22) Filed: Mar. 12, 2013

(51) Int. Cl. A61B 18/14

(52) U.S. Cl. CPC A61B 18/149
USPC 606/42

(57) ABSTRACT

Handpiece for an ablation device has a handle assembly and a tool piece with a plurality of operative elements, each operative for a different ablative purpose and for reconfiguring a handpiece for an ablation device. The handle assembly is configured to repositionably hold the tool piece in first and second orientations by the first and second portions of the handle assembly. The handpiece presents a first of the plurality of operative elements with the tool piece being positioned in the first orientation and wherein the ablation device presents a second of the plurality of operative elements with the tool piece positioned in the second orientation.

### FIELD

The present invention relates generally to handpieces for ablation devices and methods of using handpieces for ablation device and, more particularly, to handpieces for ablation devices usable for multiple purposes and methods of reconfiguring handpieces for ablation devices.

### BACKGROUND

Electrical ablation systems have been known and used for many years. William T. Bovie developed an electrosurgical unit that passes high frequency alternating electrical current into the body allowing the current to cut or coagulate.

Electrosurgery uses current or energy driven through a patient's tissue to cut or to coagulate. Two electrodes are utilized. In monopolar electrosurgery, one electrode, the device electrode, is placed in the surgical site. The second electrode to complete the circuit, sometimes referred to as the patient return electrode, is place somewhere else on the patient's body. The energy passes through the tissue of the patient as the energy completes the circuit from the device electrode to the patient return electrode. The patient return electrode is typically a conductive plate or pad, dispersive electrode, that recovers the therapeutic current from the patient during electrosurgery, dispersing the current over a wide patient area. Typically, alternating current frequencies in the RF range are utilized, sometimes approximately 490 kiloHertz.

Monopolar electrosurgery may have multiple modes of operation. To cut patient tissue, a relatively low voltage of a continuous alternating current is utilized resulting in high heat and vaporization of the tissue. Typically, the current is either one hundred percent “on” or fully “off” To coagulate patient tissue, a pulsed current of a relatively high voltage is utilized, sometimes six percent (6%) “on” and ninety-four percent (94%) off, resulting in relatively low heat and less vaporization but resulting in fulguration.

With bipolar electrosurgery, two electrodes on, or connected to, the device are the active electrodes. Unlike monopolar electrosurgery, energy generally does not travel through the patient to a distant, patient return, electrode. Instead, the energy is transferred between the two electrodes, therefore no patient return electrode is utilized. Current flows through patient tissue positioned directly between the two active electrodes. The size and shape of the electrodes affect current density through the patient tissue. A smaller surface area and sharper edges increases current density, generates greater heat and is used primarily for cutting. Electrodes with a larger and smoother surface area decreases current density, reducing the amount of heat and is commonly used for coagulation.

Transcollation is used to stop bleeding and reduce blood loss during and after surgery. Transcollation utilizes a combination of radio frequency (RF) energy and saline that provides hemostatic sealing of soft tissue and bone. Transcollation lowers transfusion rates and may reduce the need for other blood management products during or after surgery. Transcollation integrates RF energy and saline to deliver controlled thermal energy to tissue. The coupling of saline and RF energy allows the device temperature to stay at approximately 100 degrees Centigrade. The produces a tissue effect without associated charring which may otherwise be found.

Since different types of electrosurgery may be used to accomplish different purposes, ablation device handpieces typically are particularly adapted for the individual purpose being employed.

### SUMMARY

In order to accommodate differing uses of electrosurgery devices, a handpiece for an ablation device is provided that can accommodate multiple uses. One handle supports multiple uses, such as transcollation, coagulation and Bovie-style cutting. The single handle contains a tool piece with multiple operative elements. The tool piece may be manipulated in order to reconfigure the handpiece to present one operative element, e.g., transcollation, or another operative element, e.g., Bovie-style cutting, using the same tool piece and the same handle assembly.

In an embodiment, a handpiece for an ablation device has a handle assembly and a tool piece with a plurality of operative elements, each operative for a different ablative purpose. The handle assembly is configured to repositionably hold the tool piece in first and second orientations by the first and second portions of the handle assembly. The handpiece presents a first of the plurality of operative elements with the tool piece being positioned in the first orientation and wherein the ablation device presents a second of the plurality of operative elements with the tool piece positioned in the second orientation.

In an embodiment, a handpiece for an ablation device has a handle assembly having first and second elongate portions having a proximal end portion and a distal end portion, the first and second elongate portions being mated along the end portion and divergent along the distal end portion and a tool piece having a plurality of operative elements, each of the plurality of operative elements for a different ablative purpose. The distal end portions of the first and second elongate portions of the handle assembly are shaped to repositionably hold the tool in first and second orientations. The handpiece presents toward a distal end of the handle assembly a first of the plurality of operative elements with the tool piece being positioned in the first orientation. The ablation device presents a distal end of the handle assembly a second of the plurality of operative elements with the tool piece positioned in the second orientation.

In an embodiment, an ablation device has a handpiece having a handle assembly having first and second portions, a tool piece having a plurality of operative elements, each of the plurality of operative elements for a different ablative purpose and a generator selectively operatively coupled to each of the plurality of operative elements. The handle assembly is configured to hold the tool piece in first and second orientations by the first and second portions of the handle assembly. The tool piece is repositionable within the handle assembly. The handpiece presents a first of the plurality of operative elements with the tool piece being positioned in the first orientation. The ablation device presents a second of the plurality of operative elements with the tool piece positioned in the second orientation.

In an embodiment, the tool piece is locked into one of the first orientation and the second orientation by a detent on the tool and a matching projection on at least one of the first and second portions of the handle assembly.

In an embodiment, the first and second portions of the handle assembly hold the tool when the first and second portions of the handle assembly are mated with each other.

In an embodiment, the proximal portions of the first and second elongate portions of the handle assembly mate with a dovetail.

In an embodiment, the proximal portions of the first and second elongate portions of the handle assembly mate with a bayonet locking system.

In an embodiment, the first portion of the handle assembly is etched and wherein the conductive trace is plated onto the first portion of the handle assembly with a liquid crystal polymer having a semi-conductive polymer additive.

In an embodiment, the plurality of operative elements are positioned on opposite sides of the tool piece.

In an embodiment, the tool piece is disk shaped having an edge with the plurality of operative elements positioned around the edge.

In an embodiment, the disc is circular.

In an embodiment, the handle assembly is configured to hold the tool piece between the first and second portions of the handle assembly.

In an embodiment, the first and second portions of the handle assembly hold the tool when the first and second portions of the handle assembly are mated with each other.

In an embodiment, the first of the plurality of operative elements is a transcollation element having a pair of electrical contacts.

In an embodiment, the second of the plurality of operative elements is a cutting element having an electrical contact.

In an embodiment, the first portion of the handle assembly has a pair of longitudinally oriented conductive traces, the pair of conductive traces configured to be electrically coupled to the pair of electrical contacts of the transcollation element.

In an embodiment, the second portion of the handle assembly has a longitudinally oriented conductive trace configured to electrically couple to the electrical contact of the cutting element.

In an embodiment, the first portion of the handle assembly has a longitudinally oriented saline port configured to couple saline from a proximal portion of the handle assembly to the distal portion of the handle assembly.

In an embodiment, the first portion of the handle assembly is etched and wherein the conductive trace is plated onto the first portion of the handle assembly with a liquid crystal polymer having a semi-conductive polymer additive.

In an embodiment, the ablation device also has a source of saline fluidly coupled to the saline port of the handle assembly.

In an embodiment, a method reconfigures a handpiece for an ablation device for use in a different ablative purpose. A tool piece having a plurality of operative elements, each of the plurality of operative elements for one of the different ablative purpose, wherein the handpiece presents a first of the plurality of operative elements with the tool piece being positioned in the first orientation and wherein the ablation device presents a second of the plurality of operative elements with the tool piece positioned in the second orientation, is disengaged from a lock holding the tool piece in a handle assembly in a first orientation. The tool piece is repositioned in the handle assembly in a second orientation. The tool piece is engaged in the lock holding the tool piece in the handle assembly, this time in the second orientation.

In an embodiment, the tool piece is locked into one of the first orientation and the second orientation by a detent on the tool and a matching projection on at least one of the first and second portions of the handle assembly.

In an embodiment, the plurality of operative elements are positioned on opposite sides of the tool piece.

In an embodiment, the tool piece is disk shaped having an edge with the plurality of operative elements positioned around the edge.

In an embodiment, the disc is circular.

In an embodiment, the handle assembly comprises first and second portions and wherein the handle assembly is configured to hold the tool piece between first and second portions of the handle assembly.

In an embodiment, the disengaging step comprises unmating the first and second portions of the handle assembly and wherein the engaging step comprises mating the first and second portions of the handle assembly with each other.

In an embodiment, the first of the plurality of operative elements comprises a transcollation element.

In an embodiment, the second of the plurality of operative elements comprises a cutting element.

### FIGURES

FIG. 1 is an isometric view of a handpiece constructed in accordance with the present invention with a transcollation operative element presented;

FIG. 2 is an isometric view of the handpiece of FIG. 1 with a Bovie-style cutting operative element presented;

FIG. 3a illustrates a tool piece used in the handpiece of FIG. 1 from a first angle;

FIG. 3b illustrates the tool piece of FIG. 3a from a second angle;

FIG. 4 is a close-up of the distal portion of the handpiece of FIG. 1 with the transcollation operative element presented;

FIG. 5 is a close-up of the distal portion of the handpiece of FIG. 2 with the Bovie-style cutting operative element presented;

FIG. 6a, FIG. 6b and FIG. 6c are views a top portion of the handle of the handpiece of FIGS. 1 and 2;

FIG. 6d and FIG. 6e are views of a bottom portion of the handle of the handpiece of FIGS. 1 and 2;

FIG. 7 illustrates the handpiece of FIGS. 1 and 2 operatively connected to an RF generator and a source of saline solution; and

FIG. 8 is a flow chart of a method of reconfiguring a handpiece for an ablation device.

### DESCRIPTION

FIG. 1 illustrates handle assembly 10 with from top portion 12 and bottom portion 14 holding tool piece 16 at distal end 18. Top portion 12 and bottom portion 14 mate to form elongated handle assembly 10. Tool piece 16, shaped in the form of a circular disk, is rotatable, on an axis orthogonal to the major axis of handle assembly 10, between top portion 12 and bottom portion 14. Tool piece 16 carries a plurality of operative elements on its circular edge. Tool piece 16 is rotated such that transcollation element 20, consisting of first electrode 22 and second electrode 24, is presented at distal end 18 of handle assembly 10 for use as transcollation ablative elements. Tool piece 16 is held in place between arcuate end portions of top portion 12 and bottom portion 14 of handle assembly 10. Detent 26 in the outer rim of circular disk shaped tool piece 16 mates with corresponding projection 28 from bottom portion 14 to prevent tool piece 16 from rotation with top portion 12 and bottom portion mated together.

Tool piece 16 has conductive traces 30, 32 around at least a portion of the circular rim of tool piece 16 electrically coupled to first electrode 22 and second electrode 24, respectively. Conductive traces 34, 36 (not shown in FIG. 1) on the bottom side of top portion 12 extending from distal end 18 to proximal end 38 end with conductors 40, 42 electrically coupled to conductive traces 34, 36 enabling handle assembly 10 to be coupled to a source of electrical energy, for example, a source of radio frequency (RF) energy for ablative purposes. Thus, first electrode 22 and second electrode 24 may be coupled to the source of RF energy.

Tool piece 16 also carries cutting operative element 44, e.g., for Bovie-style cutting, on its circular edge roughly opposite from transcollation operative element 20. Tool piece 16 is rotated such that cutting element 20, consisting of a single electrode, is not presented at distal end 18 of handle assembly 10 for use in FIG. 1 but is available when handle assembly 10 is reconfigured for cutting. Again, tool piece 16 is held in place between arcuate end portions of top portion 12 and bottom portion 14 of handle assembly 10.

Tool piece 16 is held in place between arcuate end portions of top portion 12 and bottom portion 14 of handle assembly 10. Detent 26 in the outer rim of circular disk shaped tool piece 16 mates with corresponding projection from bottom portion 14 to prevent tool piece 16 from rotation with top portion 12 and bottom portion mated together.

FIG. 2 shows reconfigured handle assembly 10 with tool piece 16 repositioned between top portion 12 and bottom portion 14 with cutting operational element 44 being presented at distal end 18 of handle assembly for use as a cutting ablative element in conjunction with a patient return electrode (not shown). To reposition tool piece 16 in handle assembly 10, top portion 12 and bottom portion 14 may be unmated and separated from each other. In an embodiment, top portion 12 and bottom portion 14 are mated together using a commonly known dovetail arrangement. In an alternative embodiment, top portion 12 and bottom portion 14 are mated together in a commonly known bayonet locking system. Other mating and/or locking mechanisms are known in the art and contemplated as well. In order to reposition tool piece 16, top portion 12 and bottom portion 14 are at least partially unmated allowing tool piece allowing tool piece 16 to be repositioned. Top portion 12 and bottom portion 14 may be completely separated allowing tool piece 16 to be completely removed from handle assembly 10. Or top portion 12 and bottom portion 14 may be only partially separated, for example, by just allowing distal portion 18 of handle assembly to separate enough to allow tool piece 16 to be repositioned. As an example, top portion 12 and bottom portion 14 may be separated a sufficient amount to allow detent 26 to escape projection 28. Then tool piece may be rotated radially to be present cutting element 44 at the end of distal portion 18 or tool piece 16 may flipped side to side. Tool piece 16 may have a detent 26 on each side of the circular disk so that one of the two detents 26 will mate with projection 28 no matter orientation of tool piece 16 is utilized. With tool piece 16 repositioned with cutting element 44 presented and detent 26 is reengaged with projection 28, top portion 12 and bottom portion may be mated together securing tool piece 16 between top portion 12 and bottom portion 14.

In the orientation of tool piece 16 with cutting element 44 presented, conductive trace 30, electrically coupled to cutting element 44, is electrically coupled to conductive trace 48 in bottom portion 14. Conductive trace 48 may be coupled to one of conductors 40, 42 to couple cutting element 44 to a source of RF energy. Or conductive trace 48 may coupled to a separate connector (not shown) to be coupled to a source of RF energy. Alternatively, cutting element 44 may be electrically coupled to one conductive traces 34, 36 of top portion 12 rather than a separate conductive trace in bottom portion 14.

In an embodiment, handle assembly 10 may be constructed from as little as three components: top portion 12, bottom portion 14 and tool piece 16 creating an adaptable handpiece with multiple operative elements, for example, transcollation and cutting, that can be quickly and easily reconfigured.

In an embodiment, handle assembly is approximately 5.5 inches (14 centimeters) in length having a relatively straight portion approximately 3 inches (7.6 centimeters) in length. Arcuate portion at distal end 18 has a radius of curvature of approximately 0.5 inches (1.27 centimeters). The distance from the beginning of the straight portion to the point of curvature farthest from a line drawn along the straight portion is approximately 2 inches (5 centimeters). Top portion 12 is approximately 0.14 inches (0.36 centimeters) thick.

FIG. 3a and FIG. 3b illustrate tool piece 16 used in the handpiece of FIG. 1 from a first angle. Tool piece 16 is a circular disk with transcollation element 20 and cutting element 44 positioned around the circular edge or rim of tool piece 16. Recessed detent 26a is shown on one side of tool piece 16 in FIG. 3a. Corresponding detent 26b is shown on the opposite side of tool piece in FIG. 3b.

While tool piece 16 is illustrated as being a circular disk, it is to be recognized and understood that other shapes and orientations are contemplated. For example, tool piece 16 could be triangular, perhaps containing three operative elements, or square, perhaps containing two, three or four operative elements, with corresponding adaptation in mating shapes with top portion 12 and bottom portion 14 to hold tool piece 16 in position of handle assembly 10.

FIG. 4 is a close-up of distal portion 18 of handpiece 10 of FIG. 1 with transcollation operative element 20, with first electrode 22 and second electrode 24, of tool piece 16 presented. Saline duct 45 is carried by top portion 12. In an embodiment, saline duct 45 may be molded into top portion 12. In an embodiment, saline duct 45 may be constructed of tubing carried in or alongside top portion 45. It is be recognized an understood that alternative means of coupling saline from proximal portion 38 to distal portion 18 of handpiece 10 may be used, including those that are well known or which may be developed.

Conductive trace 30 and conductive trace 32 are carried by top portion 12. Conductive trace 46 is carried by bottom portion 14.

FIG. 5 is a close-up of distal portion 18 of handpiece 10 of FIG. 2 with the Bovie-style cutting operative element 44 presented. Saline duct 45 is carried by top portion 12. Conductive trace 30 and conductive trace 32 are carried by top portion 12. Conductive trace 46 is carried by bottom portion 14.

FIG. 6a, FIG. 6b and FIG. 6c are views top portion 12 of handle assembly 10 of the handpiece of FIGS. 1 and 2. Saline duct 45 runs the length of top portion 12 from proximal portion 50 to distal portion 18. Conductive trace 34 and conductive trace 36 are illustrated on the underside of top portion 12.

FIG. 6d and FIG. 6e are views of bottom portion 14 of handle assembly 10 of the handpiece of FIGS. 1 and 2. Conductive trace 46 is illustrated on the top (inside) of bottom portion 14. Projection 28 within arcuate portion of distal end 18 is illustrated in FIG. 6d. As noted in FIG. 1, FIG. 2, FIG. 4 and FIG. 5, projection 28 mates with detent 26, either detent 26a or 26b, to secure tool piece 16 from rotation or other movement with respect to top portion 12 and bottom portion 14 of handle assembly 10 with top portion 12 and bottom portion 14 mated.

FIG. 7 illustrates handpiece 10 of FIGS. 1 and 2 operatively connected to RF generator 52 and source of saline solution 54. Tubing 56 fluidly couples saline duct 45 to source of saline 54. Electrical conductors 58, 60 couple conductors 40, 44 to RF generator 52. RF generator 52 and source of saline solution 54 are conventional and are well known in the art.

FIG. 8 is a flow chart illustrating a method of reconfiguring handpiece 10 for an ablation device. Tool piece 16, while in an orientation presenting one of a plurality of operative elements, e.g. transcollation element 20, is disengaged, e.g., unmated, (810) from a lock, e.g., with detent 26 and projection 28, holding tool piece 16 in handle assembly 10. Took piece 16 is repositioned (812) in a second orientation presenting a second of the plurality of operative elements, e.g., cutting element 44. Tool piece 16 is engaged (814) between top portion 12 and bottom portion 14 of handle assembly 10, preferably by locking tool piece 16, e.g., with detent 26 and projection 28.

In an embodiment, operative elements 20, 44 are positioned on opposite sides of tool piece 16. In an embodiment, tool piece 16 is a circular disk and operative elements 20, 44 are positioned around the edge or rim of tool piece 16.

Thus, embodiments of the handpiece for an ablation device having tool piece with multiple orientations and method for reconfiguring handpiece are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A handpiece for an ablation device, comprising:
a handle assembly having first and second portions; and
a tool piece having a plurality of operative elements, each of said plurality of operative elements for a different ablative purpose;
said handle assembly being configured to repositionably hold said tool piece in first and second orientations by said first and second portions of said handle assembly;
wherein said handpiece presents a first of said plurality of operative elements with said tool piece being positioned in said first orientation and wherein said ablation device presents a second of said plurality of operative elements with said tool piece positioned in said second orientation.

2. The handpiece of claim 1 wherein said first and second portions of said handle assembly are configured to mate with each in order hold said tool piece.

3. The handpiece of claim 2 wherein said tool piece is locked into one of said first orientation and said second orientation by a detent on said tool and a matching projection on at least one of said first and second portions of said handle assembly.

4. The handpiece of claim 1 wherein said plurality of operative elements are positioned on opposite sides of said tool piece.

5. The handpiece of claim 4 wherein said tool piece is disk shaped having an edge with said plurality of operative elements positioned around said edge.

6. The handpiece of claim 5 wherein said disc is circular.

7. The handpiece of claim 1 wherein said handle assembly is configured to hold said tool piece between said first and second portions of said handle assembly.

8. The handpiece of claim 7 wherein said first and second portions of said handle assembly hold said tool when said first and second portions of said handle assembly are mated with each other.

9. The handpiece of claim 1 wherein said first of said plurality of operative elements comprises a transcollation element having a pair of electrical contacts.

10. The handpiece of claim 9 wherein said second of said plurality of operative elements comprises a cutting element having an electrical contact.

11. The handpiece of claim 10 wherein said first portion of said handle assembly has a pair of longitudinally oriented conductive traces, said pair of conductive traces configured to be electrically coupled to said pair of electrical contacts of said transcollation element.

12. The handpiece of claim 11 wherein said second portion of said handle assembly has a longitudinally oriented conductive trace configured to electrically couple to said electrical contact of said cutting element.

13. The handpiece of claim 11 wherein said first portion of said handle assembly has a longitudinally oriented saline port configured to couple saline from a proximal portion of said handle assembly to said distal portion of said handle assembly.

14. The handpiece of claim 11 wherein said first portion of said handle assembly is etched and wherein said conductive trace is plated onto said first portion of said handle assembly with a liquid crystal polymer having a semi-conductive polymer additive.

15. A handpiece for an ablation device, comprising:
a handle assembly having first and second elongate portions having a proximal end portion and a distal end portion, said first and second elongate portions being mated along said end portion and divergent along said distal end portion; and
a tool piece having a plurality of operative elements, each of said plurality of operative elements for a different ablative purpose;
said distal end portions of said first and second elongate portions of said handle assembly being shaped to repositionably hold said tool in first and second orientations;
wherein said handpiece presents toward a distal end of said handle assembly a first of said plurality of operative elements with said tool piece being positioned in said first orientation and wherein said ablation device presents a distal end of said handle assembly a second of said plurality of operative elements with said tool piece positioned in said second orientation.

16. The handpiece of claim 15 wherein said tool piece is disk shaped having an edge with said plurality of operative elements positioned around said edge.

17. The handpiece of claim 16 wherein said disc is circular.

18. The handpiece of claim 15 wherein said first of said plurality of operative elements comprises a transcollation element having a pair of electrical contacts.

19. The handpiece of claim 18 wherein said second of said plurality of operative elements comprises a cutting element having an electrical contact.

20. The handpiece of claim 19 wherein said first portion of said handle assembly has a pair of longitudinally oriented conductive traces, said pair of conductive traces configured to be electrically coupled to said pair of electrical contacts of said transcollation element.

21. The handpiece of claim 20 wherein said second portion of said handle assembly has a longitudinally oriented conductive trace configured to electrically couple to said electrical contact of said cutting element.

22. The handpiece of claim 20 wherein said first portion of said handle assembly has a longitudinally oriented saline port configured to couple saline from a proximal portion of said handle assembly to said distal portion of said handle assembly.

23. The handpiece of claim 15 wherein said proximal portions of said first and second elongate portions of said handle assembly mate with a dovetail.

24. The handpiece of claim 15 wherein said proximal portions of said first and second elongate portions of said handle assembly mate with a bayonet locking system.

25. An ablation device, comprising:
handpiece for an ablation device, comprising:
a handle assembly having first and second portions; and
a tool piece having a plurality of operative elements, each of said plurality of operative elements for a different ablative purpose;
said handle assembly being configured to hold said tool piece in first and second orientations by said first and second portions of said handle assembly;
said tool piece being repositionable within said handle assembly;
wherein said handpiece presents a first of said plurality of operative elements with said tool piece being positioned in said first orientation and wherein said ablation device presents a second of said plurality of operative elements with said tool piece positioned in said second orientation; and
a generator being selectively operatively coupled to each of said plurality of operative elements.

26. The ablation device of claim 25 wherein said plurality of operative elements are positioned on opposite sides of said tool piece.

27. The ablation device of claim 26 wherein said tool piece is disk shaped having an edge with said plurality of operative elements positioned around said edge.

28. The ablation device of claim 27 wherein said disc is circular.

29. The ablation device of claim 25 wherein said handle assembly is configured to hold said tool piece between said first and second portions of said handle assembly.

30. The ablation device of claim 29 wherein said first and second portions of said handle assembly hold said tool when said first and second portions of said handle assembly are mated with each other.

31. The ablation device of claim 25 wherein said first of said plurality of operative elements comprises a transcollation element having a pair of electrical contacts.

32. The ablation device of claim 31 wherein said second of said plurality of operative elements comprises a cutting element having an electrical contact.

33. The ablation device of claim 32 wherein said first portion of said handle assembly has a pair of longitudinally oriented conductive traces, said pair of conductive traces configured to be electrically coupled to said pair of electrical contacts of said transcollation element.

34. The ablation device of claim 33 wherein said second portion of said handle assembly has a longitudinally oriented conductive trace configured to electrically couple to said electrical contact of said cutting element.

35. The ablation device of claim 32 wherein said first portion of said handle assembly has a longitudinally oriented saline port configured to couple saline from a proximal portion of said handle assembly to said distal portion of said handle assembly.

36. The ablation device of claim 32 wherein said first portion of said handle assembly is etched and wherein said conductive trace is plated onto said first portion of said handle assembly with a liquid crystal polymer having a semi-conductive polymer additive.

37. The ablation device of claim 25 further comprising a source of saline fluidly coupled to said saline port of said handle assembly.

38. A method of reconfiguring a handpiece for an ablation device for use in a different ablative purpose, comprising the steps of:
disengaging a tool piece, having a plurality of operative elements, each of said plurality of operative elements for one of said different ablative purpose, wherein said handpiece presents a first of said plurality of operative elements with said tool piece being positioned in said first orientation and wherein said ablation device presents a second of said plurality of operative elements with said tool piece positioned in said second orientation, from a lock holding said tool piece in a handle assembly in a first orientation;
repositioning said tool piece in said handle assembly in a second orientation; and
engaging said tool piece in said lock holding said tool piece in said handle assembly, this time in said second orientation.

39. The method of claim 38 wherein said tool piece is locked into one of said first orientation and said second orientation by a detent on said tool and a matching projection on at least one of said first and second portions of said handle assembly.

40. The method of claim 38 wherein said plurality of operative elements are positioned on opposite sides of said tool piece.

41. The method of claim 40 wherein said tool piece is disk shaped having an edge with said plurality of operative elements positioned around said edge.

42. The method of claim 40 wherein said disc is circular.

43. The method of claim 38 wherein said handle assembly comprises first and second portions and wherein said handle assembly is configured to hold said tool piece between first and second portions of said handle assembly.

44. The method of claim 43 wherein said disengaging step comprises unmating said first and second portions of said handle assembly and wherein said engaging step comprises mating said first and second portions of said handle assembly with each other.

45. The method of claim 38 wherein said first of said plurality of operative elements comprises a transcollation element.

46. The method of claim 45 wherein said second of said plurality of operative elements comprises a cutting element.
